(19) United States
(12) Patent Application Publication
(10) Pub. No.: US 2011/0230723 A1
(43) Pub. Date: Sep. 22, 2011

(54) Active Instrument Port System for Minimally-Invasive Surgical Procedures

(76) Inventors: Salvatore Castro, Raleigh, NC (US); Carson Shellenberger, Raleigh, NC (US); Robert E. Welt, Wake Forest, NC (US); Daniel W. Fifer, Windsor, CA (US)

(21) Appl. No.: 12/649,307

(22) Filed: Dec. 29, 2009

Related U.S. Application Data
(60) Provisional application No. 61/141,088, filed on Dec. 29, 2008.
(60) Provisional application No. 61/229,271, filed on Jul. 28, 2009.

(51) Int. Cl. A61B 1/32

(52) U.S. Cl. 600/205

(57) ABSTRACT

An access system for surgical procedures includes an insertion tube having a distal end and a lumen, a pair of instrument delivery tubes extending through the lumen of the insertion tube, each instrument delivery tube having a fixed longitudinal position relative to the insertion tube and a steerable distal portion positioned distal to the distal end of the insertion tube. Proximal actuators are moveable to steer the distal portions of the instrument delivery tubes through manipulation of the handles of instruments extending through those tubes. First and second rigid tubes are mounted within the lumen, at least one of which has a distal end disposed within the lumen.

This application claims the benefit of U.S. Provisional Application No. 61/141,088, filed Dec. 29, 2008, and U.S. Provisional Application No. 61/229,271, file Jul. 28, 2009, each of which is incorporated herein by reference.

### FIELD OF THE INVENTION

The present invention relates to the field of access devices through which medical instruments may be introduced into an incision or puncture opening formed in a body wall.

### BACKGROUND

In conventional laparoscopic procedures, multiple small incisions or ports are formed through the skin and underlying muscle and peritoneal tissue to provide access to the peritoneal cavity for the various instruments and scopes needed to complete the procedure. The peritoneal cavity is typically inflated using insufflation gas to expand the cavity, thus improving visualization and working space. In a typical laparoscopic medical procedure, four ports are strategically placed around the abdominal area allowing the surgeon visualization and use of instruments using principles of triangulation to approach the surgical target. While this procedure is very effective and has stood as the gold standard for minimally invasive surgery, it suffers from a number of drawbacks. One such drawback is the need for multiple incisions to place the four ports, which increases the risk of complications such as post operative herniation and prolonged patient recovery. The four port method also raises concerns of cosmesis, leaving the patient with four abdominal scars.

Further developments have lead to systems allowing procedures to be performed using multiple instruments passed through a single port. In some such single port procedures, visualization and triangulation are compromised due to linear instrumentation manipulation, and spatial confinement resulting in what has been known as “sword fighting” between instruments.

An improvement on the prior single port techniques is disclosed in U.S. application Ser. No. 11/804,063 ('063 application) filed May 17, 2007 and entitled SYSTEM AND METHOD FOR MULTI-INSTRUMENT SURGICAL ACCESS USING A SINGLE ACCESS PORT (incorporated herein by reference). The '063 application describes a system for use in performing multi-tool minimally invasive medical procedures through a single instrument port into a body cavity. The system includes an expandable frame that carries a pair of tool cannulas, each of which has a lumen for receiving a tool useable to perform a procedure in the body cavity. The frame is expandable within the peritoneal cavity to orient the tool cannulas such that they allow the tools to be used in concert to carry out a procedure at a common location in the body cavity, giving the triangulation and visualization needed for optimal use of the required instruments at the operative site.

The present application describes further improvements that enhance the performance of the previously-described system.

### BRIEF DESCRIPTION OF THE DRAWINGS

FIG. 1A is a perspective view of an active instrument port system;

FIG. 1B illustrates the instrument port system of FIG. 1A in use;

FIG. 2 is a perspective view of the frame of the port system of FIG. 1A;

FIGS. 3A and 3B are perspective views of the proximal and distal ends of the insertion tube and associated features;

FIG. 4 is an exploded view of the insertion tube and features shown in FIGS. 3A and 3B;

FIG. 5 is a perspective view of the instrument delivery tubes;

FIG. 6A is a cross-section view taken along the plane designated 6A-6A in FIG. 5;

FIG. 6B is a cross-section view similar to FIG. 6A showing an alternative instrument delivery tube construction;

FIG. 7A is a perspective view of a distal end of the system of FIG. 1A, showing the instrument delivery tubes prior to deployment;

FIG. 7B is a plan view of the distal end of the system of FIG. 1A, showing the instrument delivery tubes in the deployed position;

FIG. 7C is a perspective view of the distal end of the system of FIG. 1A, with the instrument delivery tubes omitted;

FIG. 8A is similar to FIG. 7A but shows the instrument delivery tubes in the deployed position and further shows an endoscopic stapler extending from the lower rigid instrument port;

FIG. 8B is similar to FIG. 8A, but shows the instrument delivery tubes in a more closely spaced position;

FIG. 9A is a perspective view of a system of the type shown in FIG. 1A but using an alternative to the link arms and support struts;

FIG. 9B is similar to FIG. 9A but does not show the instrument delivery tubes;

FIG. 9C is a perspective view of one of the deployment springs of FIGS. 9A and 9B embodiment;

FIG. 10 is a perspective view of a proximal portion of the system of FIG. 1A, with the frame and actuation systems omitted to allow viewing of the deployment ring for the upper rigid tube;

FIG. 11 is a side elevation view showing the deployment ring and upper rigid tube of FIG. 10 and associated locking features;

FIG. 12 is a perspective view of a first alternative mechanism for advancing and retracting the upper rigid tube;

FIGS. 13A and 13B are perspective views of a second alternative mechanism for advancing and retracting the upper rigid tube;

FIG. 14 is a perspective view of the proximal portion of the frame, with the actuator systems and lid omitted, and with the instrument clamp exploded from the frame;

FIG. 15A is a perspective view showing the distal-facing portion of the instrument clamp;

FIG. 15B is similar to FIG. 15A, but omits the lever;

FIG. 16 is a perspective view of one of the instrument delivery tube actuators of the system of FIG. 1A;

FIG. 17 is an exploded view showing the control tube, proximal gimbal section, and distal section of the actuator of FIG. 16;

FIG. 18 is a perspective view showing an alternative to the actuator of FIG. 16;

FIG. 19 is a perspective view of a distal portion of the system of FIG. 1A as slightly modified to include auxiliary insufflation/smoke evacuation tubes.

FIG. 20A is a perspective view showing a clamping collar in the unlatched position before it is coupled to the spherical mount of the system of FIG. 1A.

FIG. 20B is similar to FIG. 20A but shows the clamping collar coupled to the spherical mount and placed in the latched position.

### DETAILED DESCRIPTION

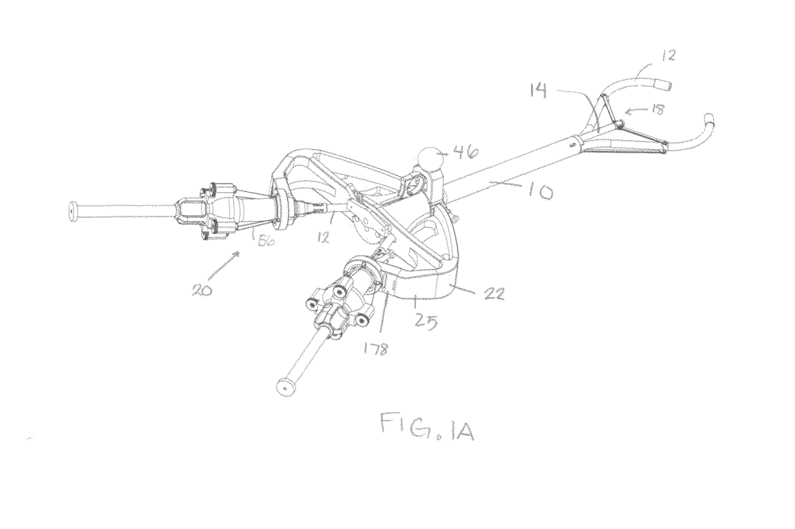

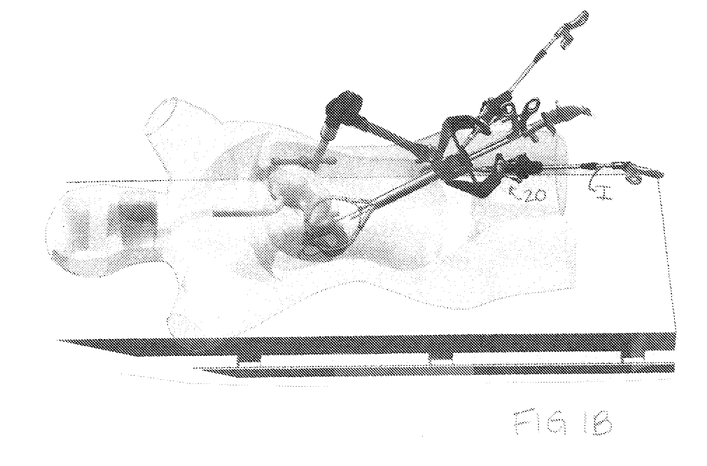

FIG. 1A illustrates one embodiment of an active port system 100. In general, the system includes an insertion tube 10 through which a plurality of access tubes extend. The access tubes comprise a combination of active access tubes and static/passive access tubes. In use, the insertion tube extends through an incision in the umbilicus or another area of a body wall such as the abdominal wall. Instruments are passed through the plurality of access tubes and used to gain access to an operative site within the body cavity. In the embodiments illustrated in the present application, the plurality of access tubes includes a pair of flexible tubes 12 having distal ends that may be deflected using proximal actuators, as well as a pair of rigid tubes 14, 16 having a fixed shape (only rigid tube 14 is visible in FIG. 1A, but see tube 16 in FIGS. 3A and 3B). In these embodiments, the distal end of rigid tube 14 is distal to the distal end of the insertion tube 10 and the distal end of the rigid tube 16 is disposed in a fixed position within the insertion tube 10.

In alternate embodiments, different combinations of tubes might be used. For example, the system might include three or more active or deflectable access tubes, and one or two passive (non-deflectable) access tubes. For example, the tube 16 might be replaced with an active tube having an actuation system similar to those used for the flexible tubes 12. The dimensions of the access tubes may vary as well. For example, in the illustrated system the flexible tubes 12 may have equal diameters (e.g. 6 mm or 12 mm) or unequal diameters (e.g. one 6 mm tube and one 12 mm tubes). The static/passive tubes may likewise have equal diameters (e.g. 6 mm each) or unequal diameters (e.g. 6 mm for the lower tube and 12 mm for the upper tube, or vice versa). While 6 mm and 12 mm are preferred diameters, other suitable diameters may instead be used.

A deployment system 18 is positioned to move the flexible tubes 12, also referred to herein as “instrument delivery tubes,” between a streamlined orientation for insertion into a body cavity and the spaced apart position shown in FIG. 1A. When in the spaced apart position, the instruments are oriented so that instruments I (e.g., graspers, dissectors, forceps, endoscopes, suture devices, staplers etc) passed through the tubes can access a common operative site as shown in FIG. 1B. Actuators 20 provided on the proximal portion of the system can be manipulated to deflect the distal ends of the instrument delivery tubes using a pullwire tensioning system. This allows simple flexible instruments, rather than those having sophisticated articulation features, to be used in the instrument delivery tubes, since the instrument delivery tubes are themselves deflected in order to move or reposition the corresponding instruments. The system components are supported by a base/support disposed outside the patent and having a coupling that mounts the base to a fixture 200 within the operating room. In the illustrated embodiment, components of the base include a frame 22 and an attachment bracket 36, although various types of bases/supports may be alternatively used.

Frame

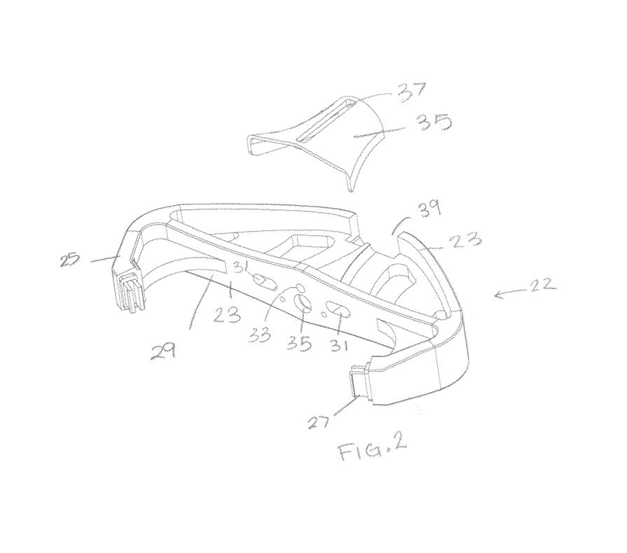

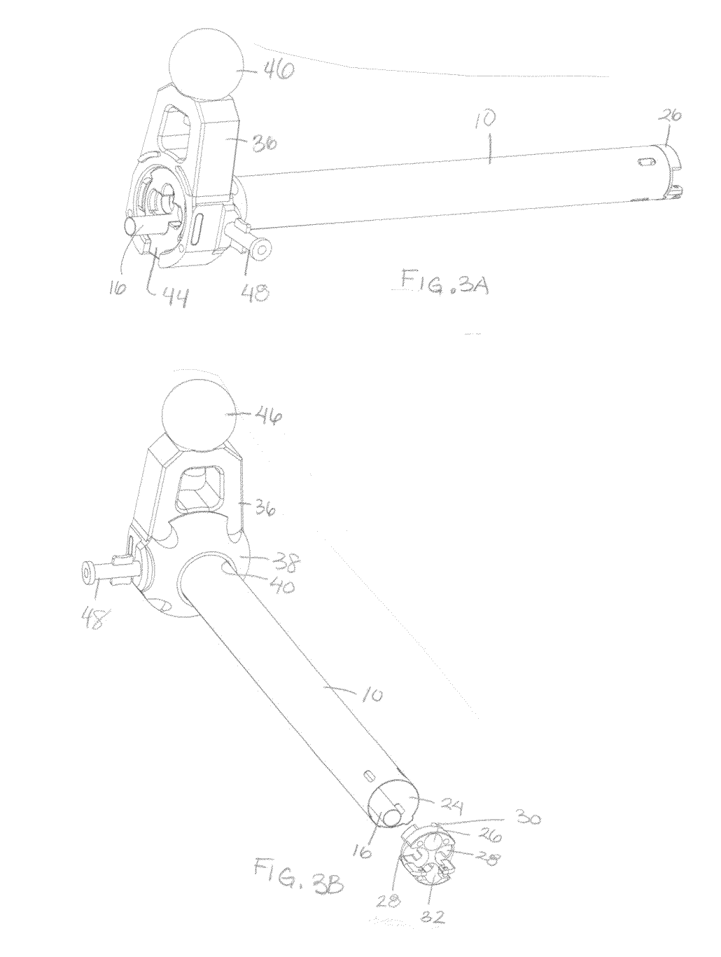

FIG. 2 shows the frame 22 with the various components removed from it. A preferred frame is formed of injection molded plastic, but it may also be formed from aluminum or other materials.

The frame 22 has a generally C-shaped outer wall 23. The free ends 25 of the “C” are positioned on the proximal side of the frame 22 and include mounts 27 for supporting the actuators 20 (not shown in FIG. 2, see FIG. 1A). A panel 29 positioned distal to the free ends 25 extends laterally between portions of the C-shaped wall 23. Openings 31 in the panel 29 are positioned to receive proximal sections of the flexible tubes 12, while other openings 33, 35 are positioned to receive instruments extending into the rigid tubes 14, 16. The distal portion of the frame includes a cutout 39.

Reinforcing web sections 37a extend between the panel 29 and the wall 23 on the distal side of the wall. Web sections 37b also extend between the panel and the wall on the proximal side. A lid 35, shown exploded from the frame in FIG. 2, is positionable between the panel 29 and the cutout 39 as shown in FIG. 1A, creating a hollow region. The lid 35 includes a longitudinal slot 37.

Many of the figures that follow show various system components separate from the frame and/or surrounding components to permit their features to more easily be seen and understood.

Insertion Tube

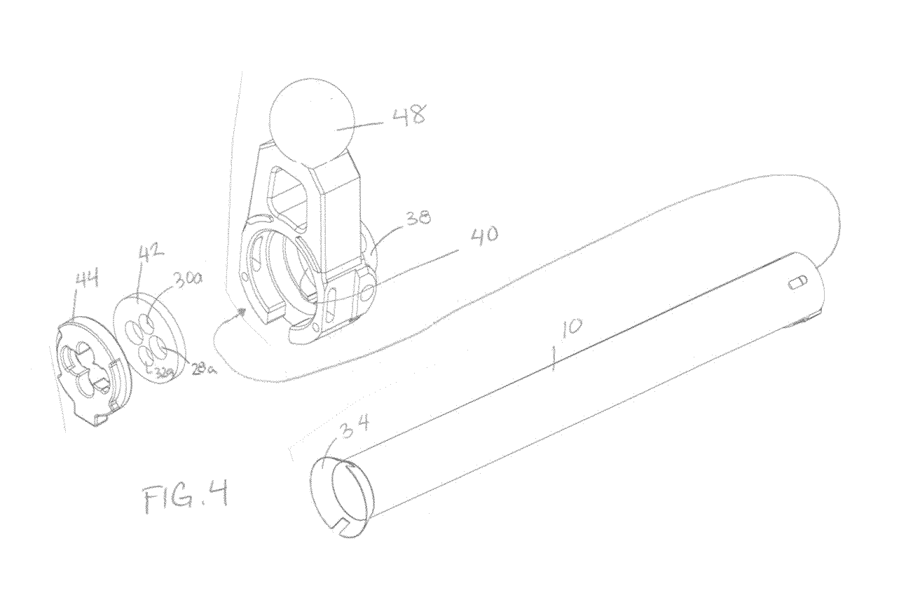

FIGS. 3A-4 show the insertion tube 10 and associated components separated from the overall system.

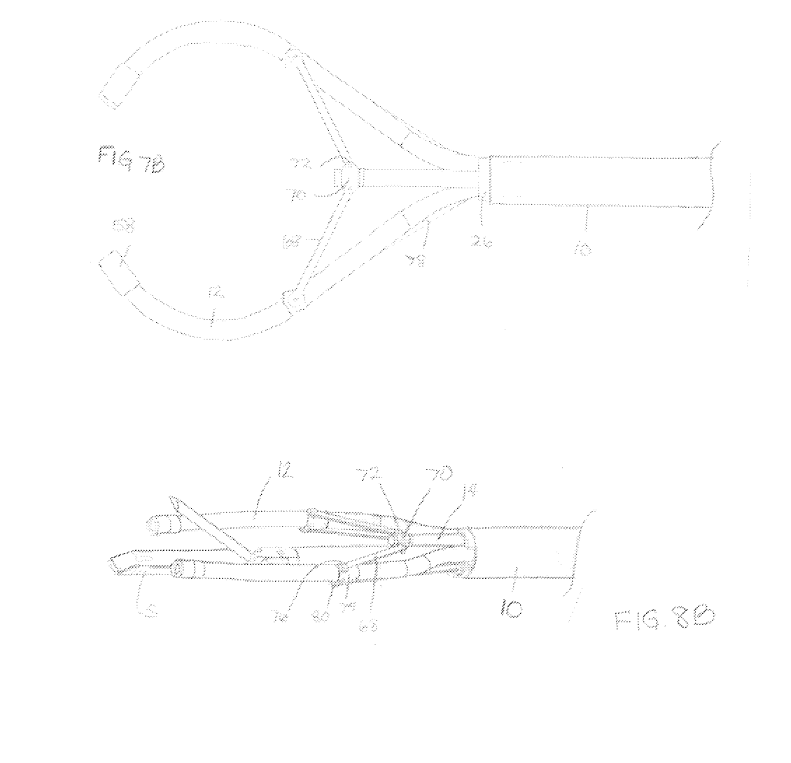

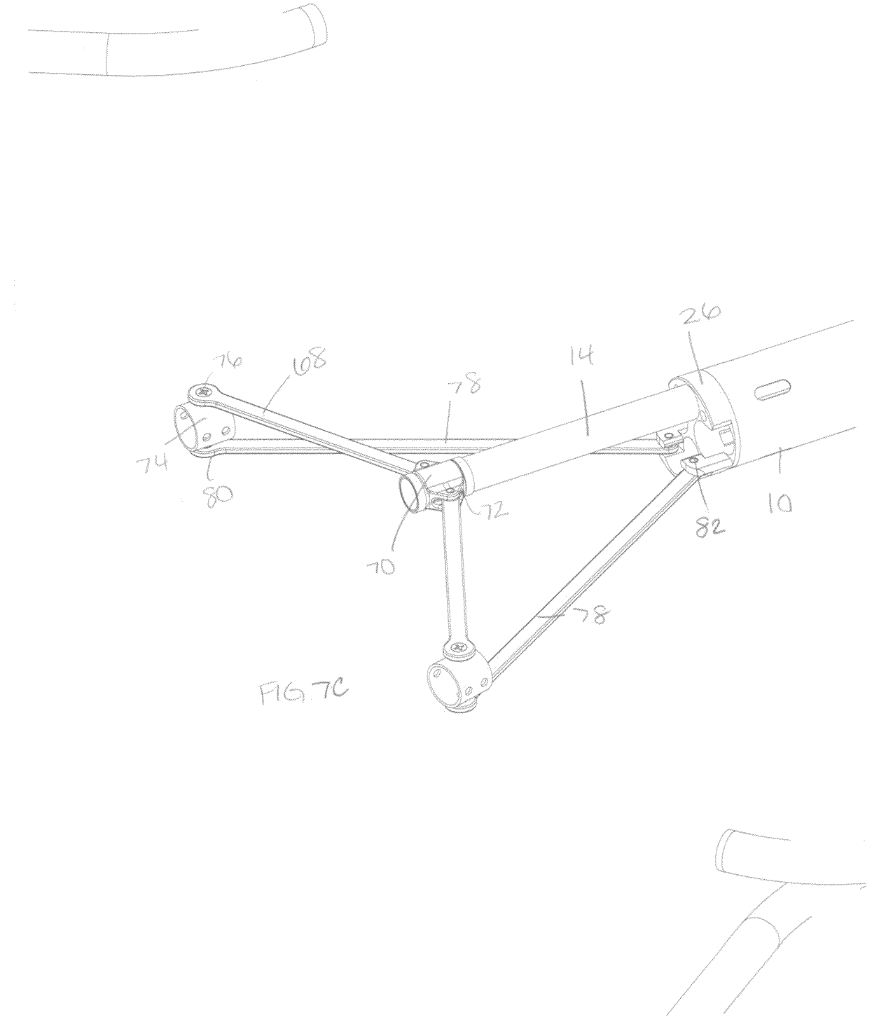

Referring to FIGS. 3A and 3B, the insertion tube 10 is an elongate rigid tube formed of stainless steel or other suitable rigid material. The insertion tube 10 preferably has a single lumen 24 proportioned to accommodate the two instrument delivery tubes 12 and the rigid tubes 14, 16. For simplicity, only the rigid tube 16 is shown in FIGS. 3A and 3B, although the other tubes 12, 14 are visible in FIGS. 1A, 7A, 7B and elsewhere in the drawings.

The insertion tube 10 includes a guide at its distal end that serves to maintain the relative lateral positioning of instruments extending through the insertion tube. The guide may be any structure that helps to prevent lateral movement of instruments within the insertion tube. As best shown in FIG. 3B, in a preferred embodiment the guide takes the form of a plate 26 defining openings 28, 30, 32. In a preferred arrangement shown in FIG. 3B, a pair of the openings 28 are positioned side-by-side, with another opening 30 centrally positioned above the pair 28 and a fourth opening 32 centrally positioned below the pair of openings 28. When the system is assembled, instrument delivery tubes 12 extend through the openings 28, and instruments inserted through rigid tubes 14, 16 extend through the openings 30 and 32, respectively.

Referring to FIG. 4, the proximal end of the insertion tube 10 includes an outwardly flared skirt section 34. An attachment bracket 36 coupled to the proximal end of the insertion tube 10 includes a tapered nose section 38 (FIG. 3B). During assembly, the insertion tube 10 is inserted through the lumen 40 of the attachment bracket (FIG. 4) in a distal direction so that its outwardly flared skirt section 34 seats within the nose section 38 of the attachment bracket 36.

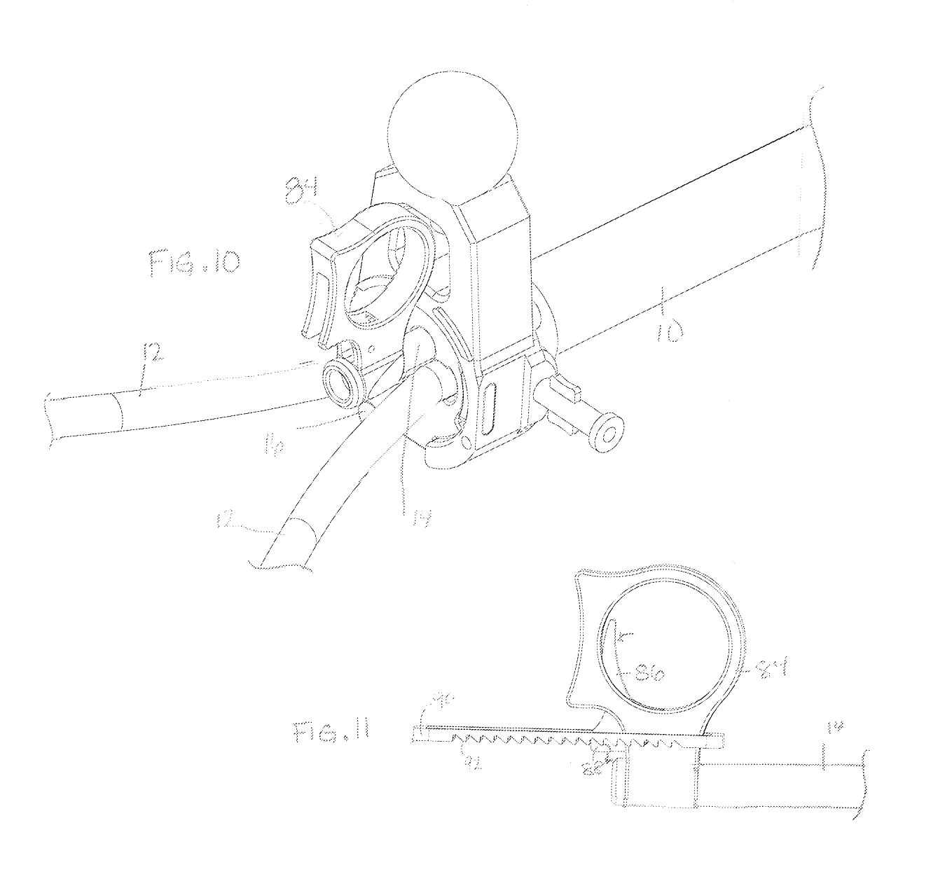

A gasket 42 is positioned within the lumen 40 of the attachment bracket. The gasket 42 includes openings 28a, 30a, and 32a that are longitudinally aligned with the openings 28, 30, 32 of the distal plate 26 (FIG. 3B). A proximal compression plate 44 compresses the gasket 42 within the lumen 40 of the attachment bracket in a distal direction. When the system is assembled, the tubes 12, 14, 16 extend through corresponding ones of the openings 28a, 30a, 32a (see also FIG. 10), which make sealing contact against the shafts of the tubes 12, 14, 16 so as to minimize loss of insufflation pressure within the abdominal cavity.

The attachment bracket may optionally include a luer valve 48 which can be fluidly coupled to a source of insufflation gas for inflating the abdominal cavity through the insertion tube 10. The valve 48 may also be coupled to a vacuum source for use in evacuating smoke from the abdominal cavity during electrosurgical or other thermal procedures. The valve may also be opened to passively evacuate smoke from the abdominal cavity.

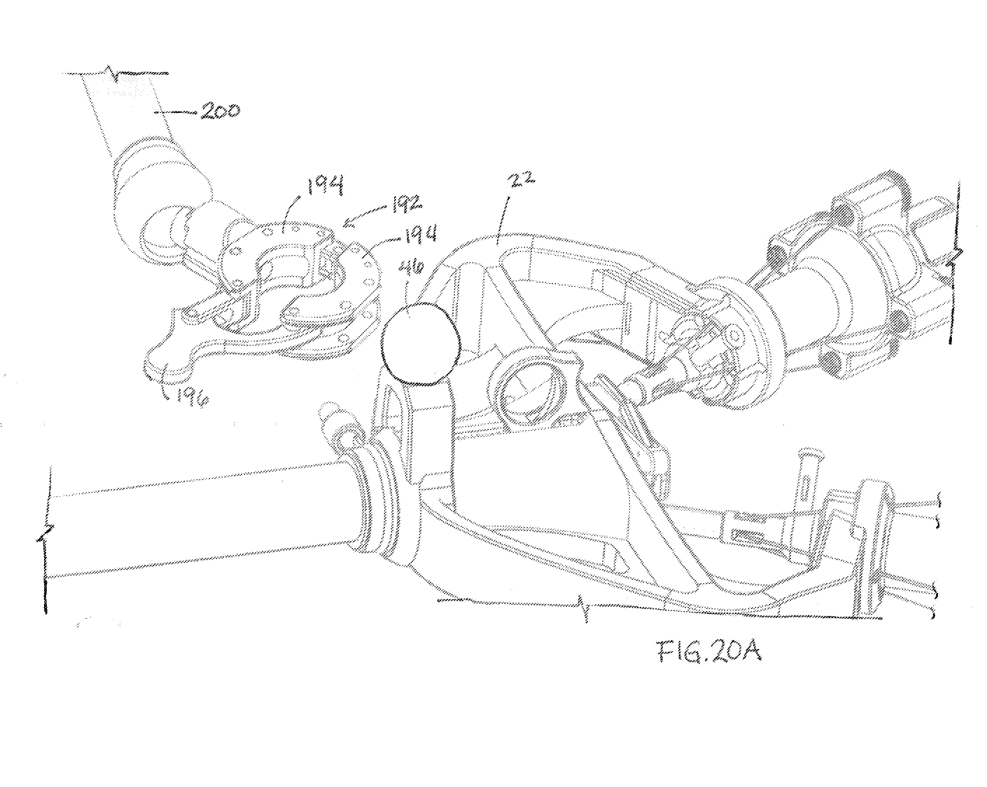

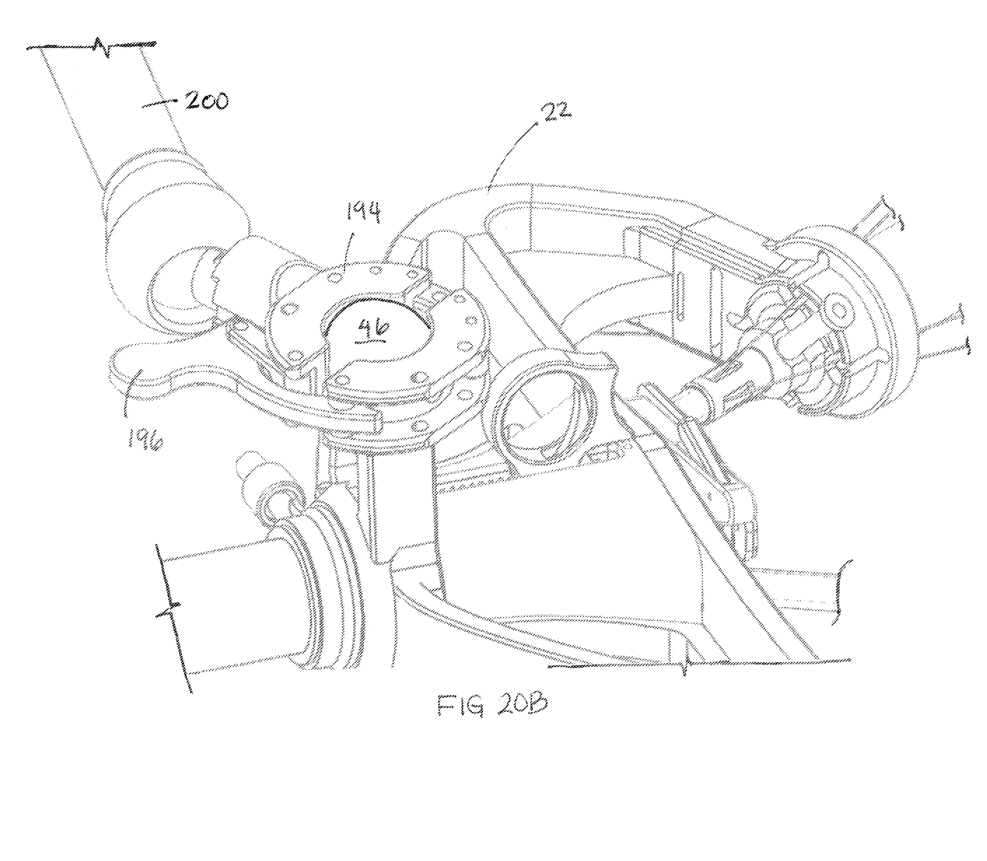

When the system is assembled, the attachment bracket 36 is mounted to the frame in cutout 39 (FIG. 2), thereby coupling the insertion tube 10 to the frame 22 as can be seen in FIG. 1A. A mount 46 on the attachment bracket 36 is used to attach the system 100 to an operating room fixture as will be discussed in greater detail in connection with FIGS. 20A and 20B.

Instrument Delivery Tubes

Referring to FIG. 5, each instrument delivery tube 12 includes a proximal end section 50, an intermediate section 52, and a distal section 54. The tube 12 can have any desired shape. For example, in one embodiment the tube 12 may have a generally straight shape through the proximal, intermediate and distal section.

In alternate embodiments, different sections of the tube 12 may have preformed shapes. For example, in one such alternate embodiment, distal section 54 has a pre-shaped curve that generally curves outwardly from the intermediate section 52 and then inwardly. The curvature of the distal sections 54 serves to orient the distal sections 54 towards one another such that instruments passed through the instrument delivery tubes 12 can access a common treatment site.

In the assembled system, the intermediate sections 52 are disposed within the insertion tube 10. Thus, these sections extend generally linearly in parallel to one another as shown. The proximal end sections 50 are disposed outside the proximal end of the insertion tube, and flare away from the longitudinal axis of the main tube (FIG. 1A) to allow their corresponding actuators 20 to be spaced a comfortable distance apart on the frame 22. This facilitates a similar to feel to laparoscopic procedures.

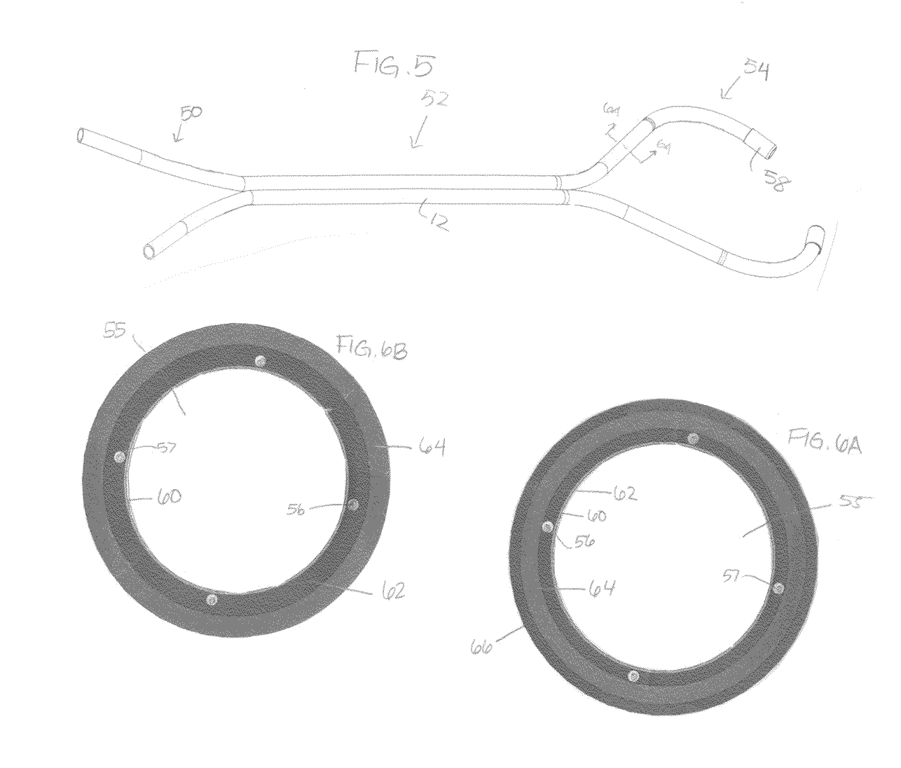

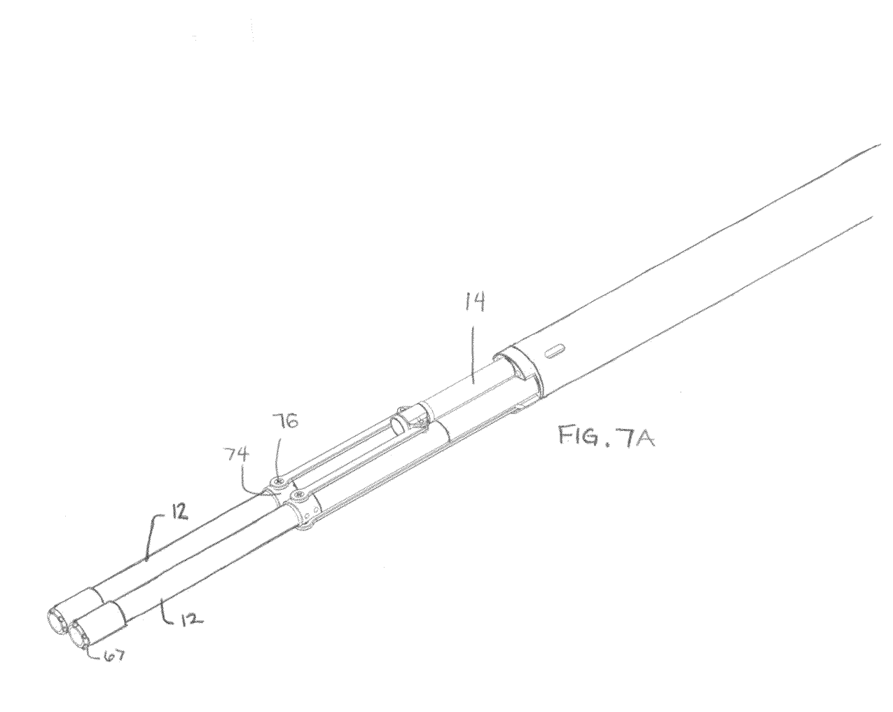

Referring to the cross-section view of FIG. 6A, each instrument delivery tube includes a lumen 55 and a plurality of pull wires 56 extending through pullwire lumens 57 and anchored with the distal end section 54. In the preferred embodiment, each instrument delivery tube has four wires such arranged at 90 degree intervals. Other embodiments can utilize different numbers of pullwires, such as three pullwires equally spaced around each instrument delivery tube 12.

As will be discussed in detail below, the set of pullwires for each of the instrument delivery tubes 12 is coupled to a corresponding actuator 20, which may be manipulated to deflect the distal end sections 54. By deflecting the instrument delivery tubes, the flexible instruments extending through them are deflected within the body into desired positions and orientations.

The instrument delivery tubes are constructed to be sufficiently flexible to allow the required deflection for instrument manipulation, while also being resistant to kinking. In one embodiment, each instrument delivery tube includes a PFTE inner liner 60 lining the lumen 55, a thermal plastic sheath 62 (having the pull wire lumens 57 formed through it) overlaying the liner 60, a reinforcing layer 64 over the thermal plastic sheath 62, and a second thermal plastic sheath 66 over the reinforcing layer. In a variation shown in FIG. 6B, the second thermal plastic sheath is eliminated and the reinforcing layer 64 serves as the outer layer of the sheath. In yet another embodiment, the reinforcing layer may comprise the most inner layer of the tube. Various other embodiments, including those having additional layers of reinforcing material or other materials can also be used.

In some embodiments, the instrument delivery tubes may be provided with illumination sources used to illuminate the surgical site within the body. For example, optical fibers or light emitting diodes 67 (FIG. 7A) may be carried by the distal ends of the instrument delivery tubes and coupled to an energy supply for energizing the illumination source. The energy supply might be one or more batteries carried by the system, or the external power source available within the surgical theatre.

Deployment System

The deployment system 18 is operable to reposition the distal portions of the instrument delivery tubes 12 to increase or decrease the distance between them. In particular, the deployment system 18 can move the instrument delivery tubes 12 between the closely spaced position shown in FIG. 7A and the more widely spaced (and, optionally, inwardly oriented) position shown in FIG. 7B.

The deployment system 18 includes a rigid tube 14 and a pair of link arms 68. The rigid tube 14 is preferably a straight, single-lumen, tube made of stainless steel or rigid polymeric material. The rigid tube 14 serves the dual role of (1) providing access into the body cavity for rigid or flexible instruments or scopes and (2) controlling adjustment of the lateral spacing of the instrument delivery tubes 12 for deployment and/or repositioning.

Each link arm 68 has a first end pivotally coupled to the rigid tube 14 and a second end pivotally coupled to a corresponding instrument delivery tube 12, proximally of its distal end. The rigid tube 14 is longitudinally moveable relative to the insertion tube 10 to pivot the link arms 68 inwardly and outwardly. In the illustrated configuration, sliding the tube 14 in a distal direction pivots the link arms 68 outwardly from the position shown in FIG. 7A to the position shown in FIG. 7B to deploy or further separate the instrument delivery tubes 12. Alternate configurations may operate in reverse, so that retraction of the rigid tube 14 increases the separation of the instrument delivery tubes.

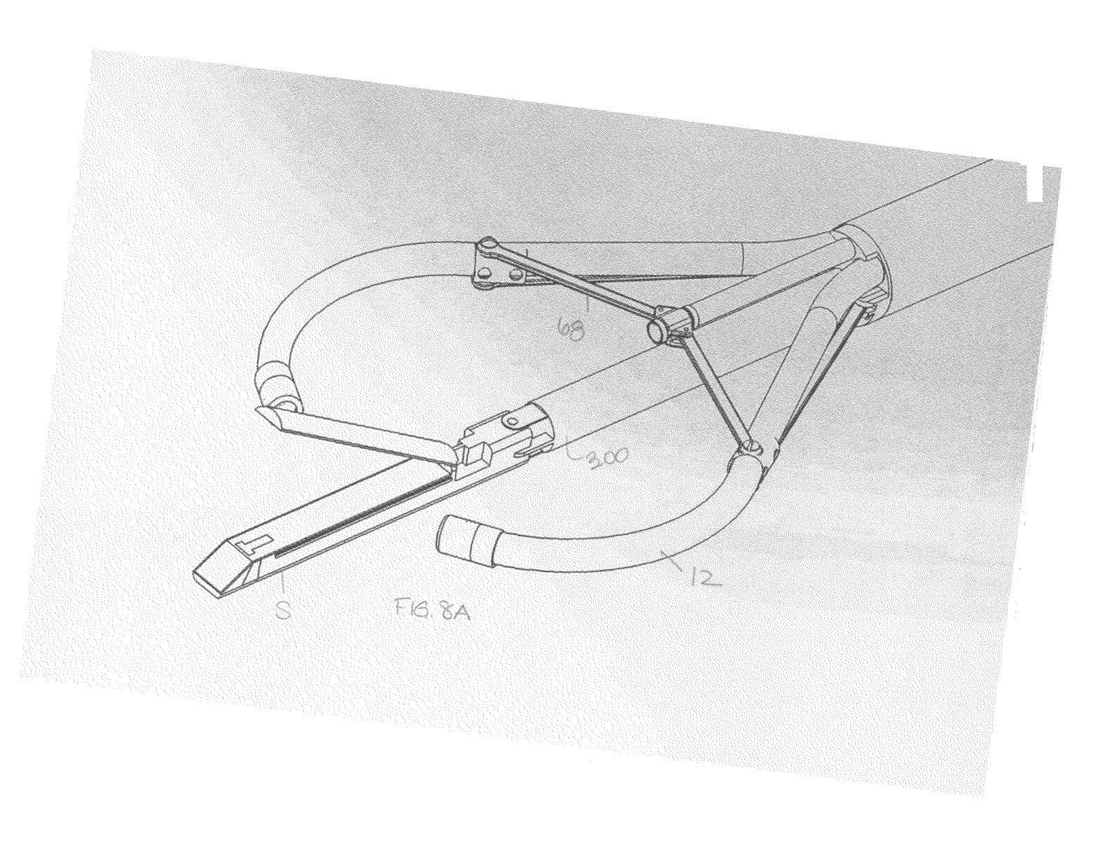

In the embodiment of FIGS. 7A-8B, the rigid tube 14 includes a tubular collar 70 having hinge pins 72 to which the link arms 68 are coupled. Each instrument delivery tube 12 may similarly include a tubular collar 74 and pins 76 for forming a pivot connection as shown.

As best shown in FIG. 7C in which the instrument delivery tubes 12 are omitted, support members or struts 78 are pivotally connected to second pins 80 on the collar 74 and extend to hinge pins 82 on the plate 26. The proximal ends of the struts 78 are pivotally coupled to the spaced apart hinge pins 82 on opposite sides of the lower opening 32. The pins 76, 80 coupling the links 68 and struts 78 for a particular tube 12 are axially aligned so that the distal ends of a given link-strut pair pivot relative to a common pivot axis.

The support struts 78 support the instrument delivery tubes 12, helping to maintain the longitudinal orientation of the instrument delivery tubes 12, and preventing the tubes 12 from sagging or buckling during use. The support struts 78 preferably extend directly beneath the portions of the IDT's that extend between the collars 74 and the insertion tube 10 so that they do not obstruct visualization or use of instruments.

In a preferred arrangement, the ends of the link arms 68 that are coupled to the instrument delivery tubes 12 remain distal to the ends that are coupled to the rigid tube 14, both when the instrument delivery tubes are in the closely spaced position of FIG. 7A and when they are in the fully expanded position of FIG. 7B. With this arrangement, the most centrally positioned end of the link arm always extends in a proximal direction, away from the operative site. This arrangement positions the link arms away from the working space and minimizes the likelihood that they will interfere with use of instruments (such as an endoscopic stapler S having jaws with a large sweep) passed through the lower rigid tube 16 as shown in FIGS. 8A and 8B. An open working space is further optimized by coupling the link arms to the rigid tube 14 at its distal tip section, so that the rigid tube 14 does not extend so far distally as to obstruct the use of instruments passed through the lower rigid tube 16. To further minimize conflict between instruments and features of the deployment system, the distal tip of the rigid tube 14 preferably has a distalmost position that is proximal to the distalmost positions of the instrument delivery tubes.

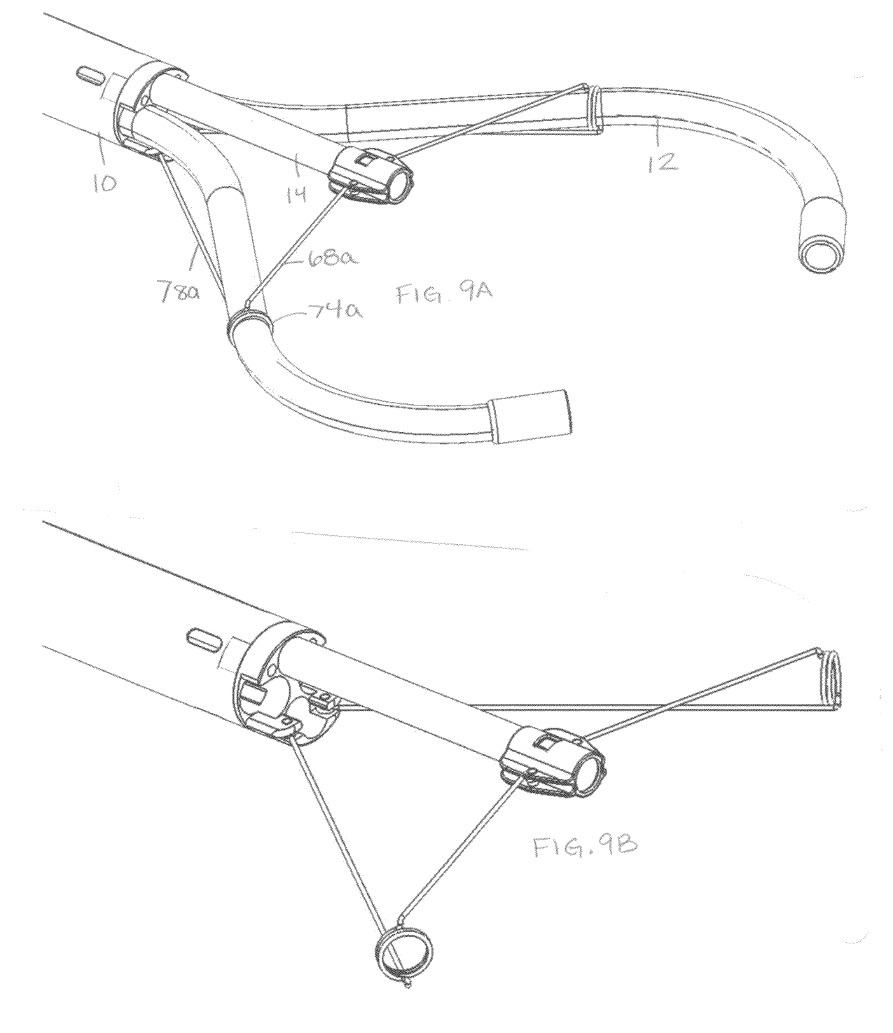

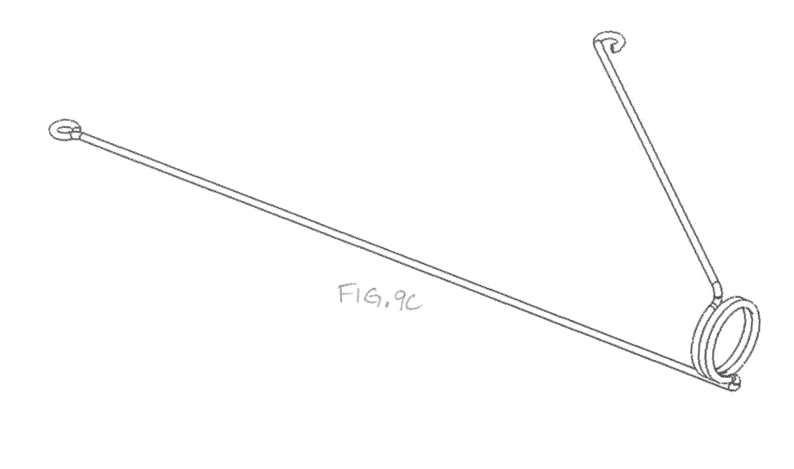

In an alternative embodiment shown in FIG. 9A, the link arm 68, support strut 78 and collar 74 associated with each instrument delivery tube 12 is replaced by a stainless steel or nitinol spring member such as a torsion spring. Each such torsion spring includes a coil section 74a coupled to (e.g. disposed around) the instrument delivery tube 12, a first leg 68a extending from the coil section 74a and coupled to the rigid tube 14, and a second leg section 78a extending from the coil section 74a and coupled to the insertion tube 10. As with the prior embodiment, the instrument delivery tubes are deployed by advancing the rigid tube 14 distally to pivot the torsion springs outwardly.

Various mechanisms may be provided for sliding the rigid tube 14 longitudinally for instrument delivery tube deployment and spacing adjustment. As shown in FIG. 10, the proximal end of rigid tube 14 extends out of or is positioned proximally of the proximal end of the insertion tube 10 and the base (e.g. attachment bracket 36). In some embodiments, the user may directly grasp the proximal portion of the rigid tube 14 or use a push/pull slider manipulator or other handle to advance or retract it longitudinally. Alternatively, as illustrated in FIG. 10, the user may advance/retract the rigid tube 14 by advancing/retracting a ring 84 coupled to the rigid tube 14. In the assembled system, the ring 84 extends through the longitudinal cutout 37 in the lid 35 (see FIGS. 1A and 2), allowing the user easy access to the ring using an index finger.

A preferred deployment system allows full deployment of the instrument delivery tubes into the position shown in FIG. 7B, but also allows the user to position the tubes at smaller separation distances (e.g. the position shown in FIG. 8B) as appropriate for a given step in the operative procedure. Using the illustrated embodiment, the user may choose to fully advance the ring 84 for full deployment, or to only partially advance the ring 84 for less than full deployment of the instrument delivery tubes. In some procedures, the user might use the ring 84 to reposition the instrument delivery tubes multiple times during a procedure to increase or decrease the separation between the tubes as needed at a given moment.

The deployment system includes a locking mechanism usable to lock the rigid tube 14 at a given longitudinal position so as to prevent an unintended change in the separation distance between the instrument delivery tubes. In the FIG. 10-11 embodiment, the ring 84 includes a pivotable trigger 86 having a catch 88. A ratchet plate 90 having two parallel rows of teeth 92 is mounted within the lid 35 (FIG. 2). A spring (not shown) between the trigger 86 and the ring 84 biases the catch in engagement with a corresponding pair of teeth 92 in the ratchet 90. Depressing the trigger 86 disengages the catch 88 from the teeth 92, allowing the ring to be moved distally or proximally to reposition the rigid tube 14. When the rigid tube 14 has been repositioned to give the instrument delivery tubes the desired amount of separation, the trigger 86 is released, allowing the spring bias to pivot the catch 88 into engagement with another pair of teeth 92 of the ratchet, thus locking the longitudinal position of the rigid tube 14 and fixing the lateral separation distance between the instrument delivery tubes.

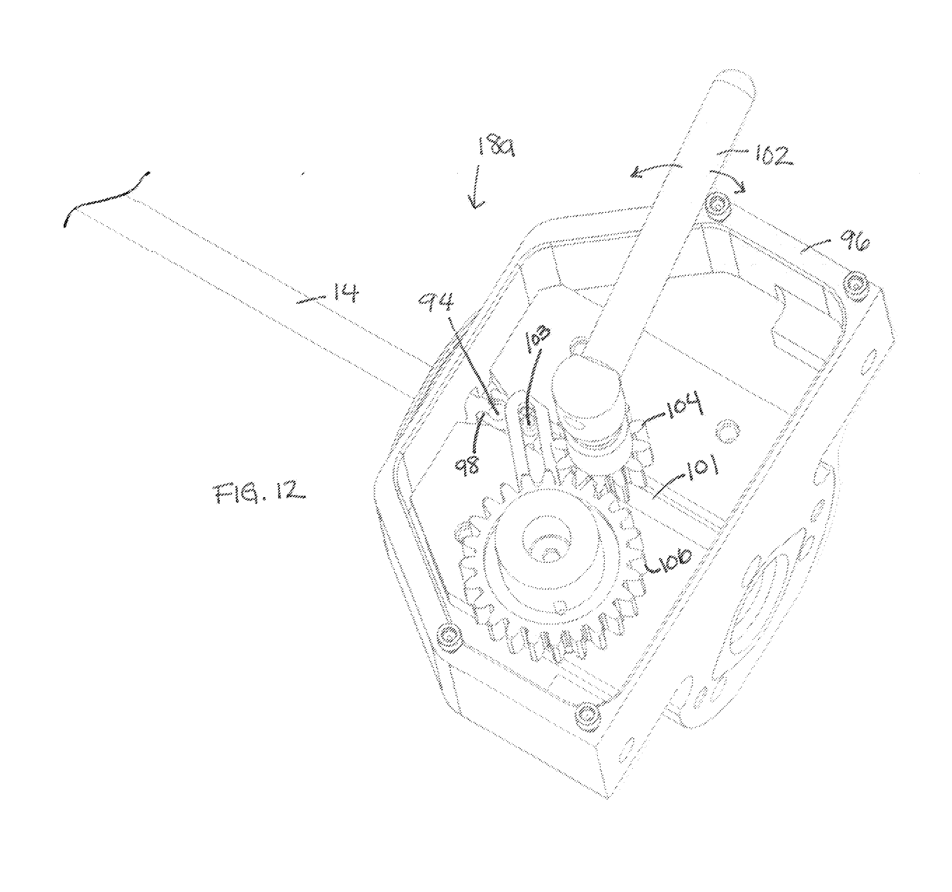

FIG. 12 shows an alternate deployment system 18a for advancing the rigid tube 14 so as to deploy the link arms. In this embodiment, a catch 94 is mounted to the proximal end of the rigid tube 14. The proximal end of the tube extends into a gear box 96, and the catch 94 is engaged to a clip 98 within the gear box. The clip 98 is coupled to a pin 103. The clip 98 is longitudinally slidable within a track 101 in the gear box.

A control lever 102 rotates a corresponding gear 104 within the gearbox. The teeth of gear 104 engage the teeth of a second gear 106. A camming lever 110 extends from the axis of the second gear 106, such that as the second gear 106 rotates, the camming lever pivots about the axis of the second gear. The pin 103 is disposed within the opening of the camming lever.

To longitudinally advance or retract the rigid tube 14, a user pivots the control lever 102, thus causing the gear 104 to rotate. Rotation of the gear 104 drives rotation of the second gear 106, and thus causes the camming lever 110 to pivot. As the camming lever 110 pivots, it cams the pin 103 in a distal or proximal direction within the slot 101, thus advancing the rigid tube 14 in a proximal or distal direction. Following use of the system, the rigid tube 14 may be detached from the gear box 104 by pulling the rigid tube 14 away from the gear box to separate the catch 94 from the clip 98. This allows for sterilization and subsequent re-use of the rigid tube 14 and/or gear box 96.

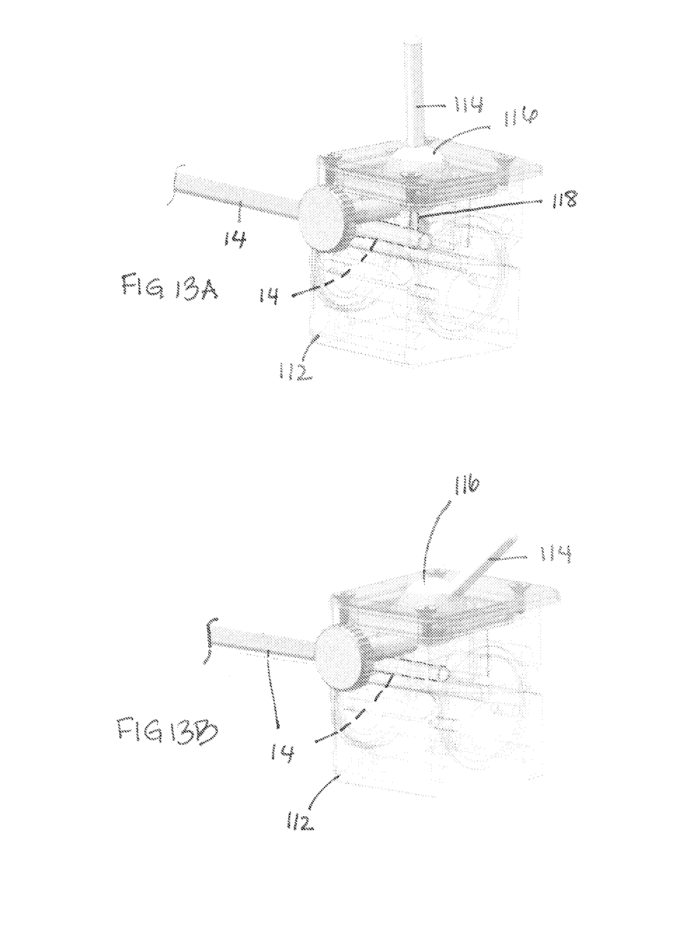

FIGS. 13A and 13B show yet another deployment system 18b. In this embodiment, the proximal end of rigid tube 14 extends into a housing 112. A control lever 114 is mounted on spherical swivel 116 seated in housing 112. A second, internal, lever 118 extends from the spherical swivel 116 and is coupled to the rigid tube 14 within the housing, such that actuation of the control lever 114 in a proximal or distal direction pivots the internal lever 118 in a distal or proximal direction, thus advancing or withdrawing the rigid tube 14.

Instrument Clamp

As discussed, in addition to its function as a deployment actuator for the link arms, the rigid tube 14 is designed to function as a port to accommodate passage of an instrument or scope into the body cavity. The system preferably includes features allowing the longitudinal position of those instruments to be selectively fixed, while still allowing longitudinal movement of the rigid tube 14 when the lateral spacing between the instrument delivery tubes needs adjusting. The attached drawings show one form of clamp provided on the base and moveable to a closed position to clamp against the outer surface of the rigid tube 14, however it should be appreciated that many alternative clamp designs may instead be used.

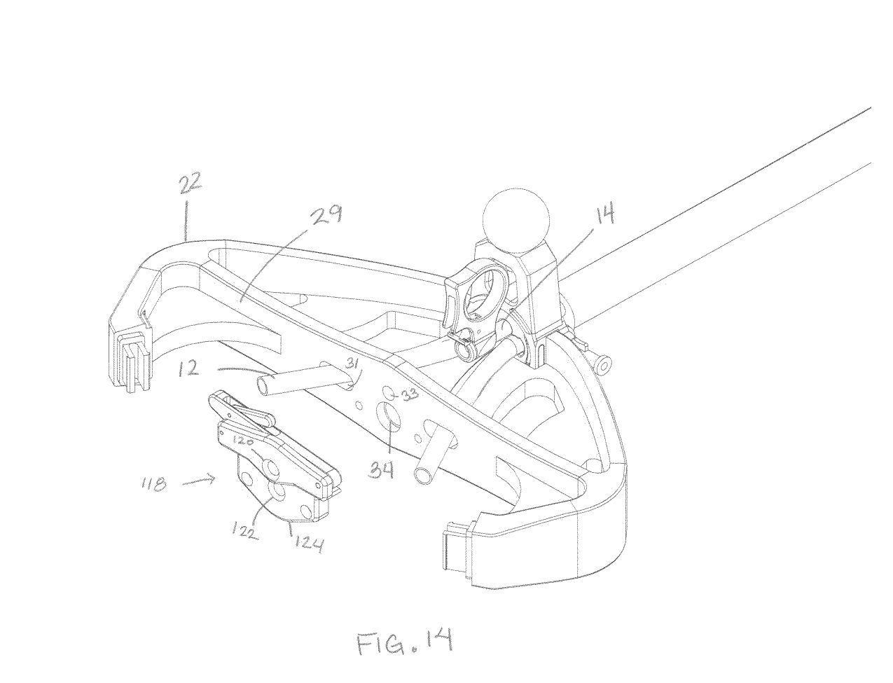

Referring to FIG. 14, the lateral panel 29 of the frame 22 includes an upper hole 33 longitudinally aligned with the upper rigid tube 14 and a lower hole 34 longitudinally aligned with the lower rigid tube 16 (not visible in FIG. 14). An instrument clamp assembly 118, shown exploded from the frame 22, has a housing 124 including upper and lower holes 120, 122 longitudinally aligned with the holes 33, 34 in the frame. Thus, an instrument to be inserted into the upper rigid port 14 is inserted into the hole 120 of the housing, and passes through the upper hole 33 of the frame 22 and into the rigid tube 14. Similarly, an endoscope or instrument to be used through the lower rigid tube extends through the lower ports 34, 122 of the housing and frame and into the lower rigid tube 16.

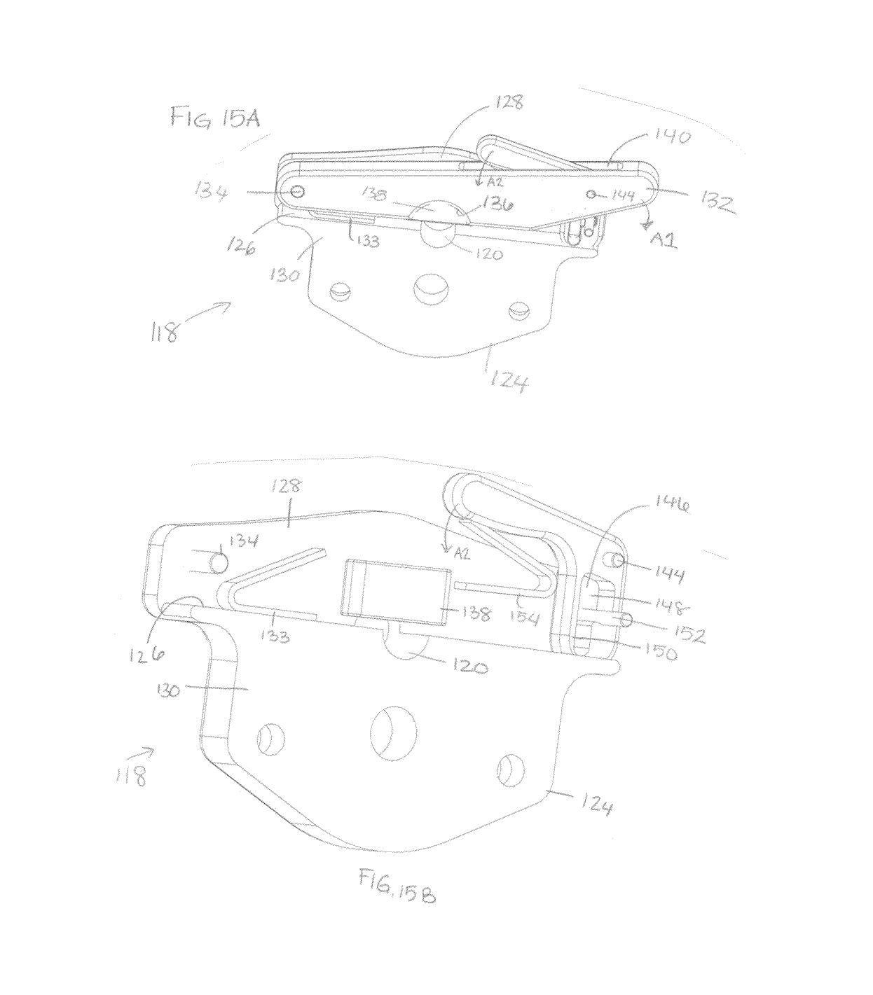

Reference is now made to FIG. 15A, which shows the distal side of the clamp assembly 118 which in the assembled system faces the panel 29 of the frame 22. As shown, the housing 124 includes a relatively thinner upper section 128 forming a shelf 126 with the relatively thicker lower section 130. The plane of the shelf 126 transects the upper hole 120, such that an upper portion of the hole 120 passes through the upper section 128 and a lower portion of the hole passes through the lower section 130.

A lever 132 is pivotally coupled by a pin 134 to the distal face of the housing 124 at the upper section 128. The lever 132 includes a semi-circular cutout 136 along its lower edge, facing the shelf 126 and at least partially aligned with the upper hole 120. A clamp insert 138, formed of silicone or other flexible material, is disposed within the lever 132 and covers the cutout 136. The lever is pivotable between a first position in which the clamp insert is rotated out of alignment with the upper hole 120 and a second position in which the clamp insert at least partially eclipses the upper hole 120. A spring 133 is positioned between the shelf 126 and a portion of the lever (e.g. a wall within the lever) and biases the lever in the first (non-clamping) position.

The lever 132 also includes a slot 140. A clamp clip 142 is disposed in the slot 140 and is pivotally coupled to the lever 132 by a pin 144. FIG. 15B, which is similar to FIG. 15A but shows the assembly without the lever 132, illustrates features of the clamp clip 142. As shown, clamp clip 142 includes a cutout 146 having a wider upper section 148 and a relatively narrow lower section 150. A dowel 152 is disposed within the cutout. A spring 154 extends between the clamp clip 142 and a portion of the lever within the slot 140 (FIG. 15A).

The clamp assembly 118 functions to secure an instrument positioned in the rigid tube 14 by clamping against the portion of the instrument's shaft that is disposed in the upper hole 120. To use this feature, the user depresses the lever 132, pivoting it as indicated by arrow A1 in FIG. 15A causing the clamp insert 138 to clamp the instrument shaft against the lower portion of the hole 120. To maintain the clamped position, clamp clip 142 is pivoted around pin 144 as indicated by arrow A2. As the clamp clip 142 pivots, the cutout 146 is reoriented such that the dowel 152 seats within the narrow portion 150 of the cutout 146, locking the lever 132 in the clamping position to thereby maintain clamping force against the instrument shaft. When the longitudinal position of the instrument is to be changed, or when the instrument is to be removed from the rigid tube 14, the clamp clip 142 is moved from the latched position to an unlatched position, allowing the lever 132 to return to its original position and thereby unclamping the instrument shaft.

Instrument Delivery Tube Actuators

As previously described, the instrument delivery tubes 12 are provided with pullwires that are activated to deflect the distal portions of the instrument delivery tubes, allowing deflection of the flexible instruments disposed within them. Referring again to FIG. 1A, the proximal portions of the instrument delivery tubes are coupled to actuators 20 that are manipulated by a user to activate the pullwires 56. The actuators 20 are mounted to the proximal ends 25 of the frame 22.

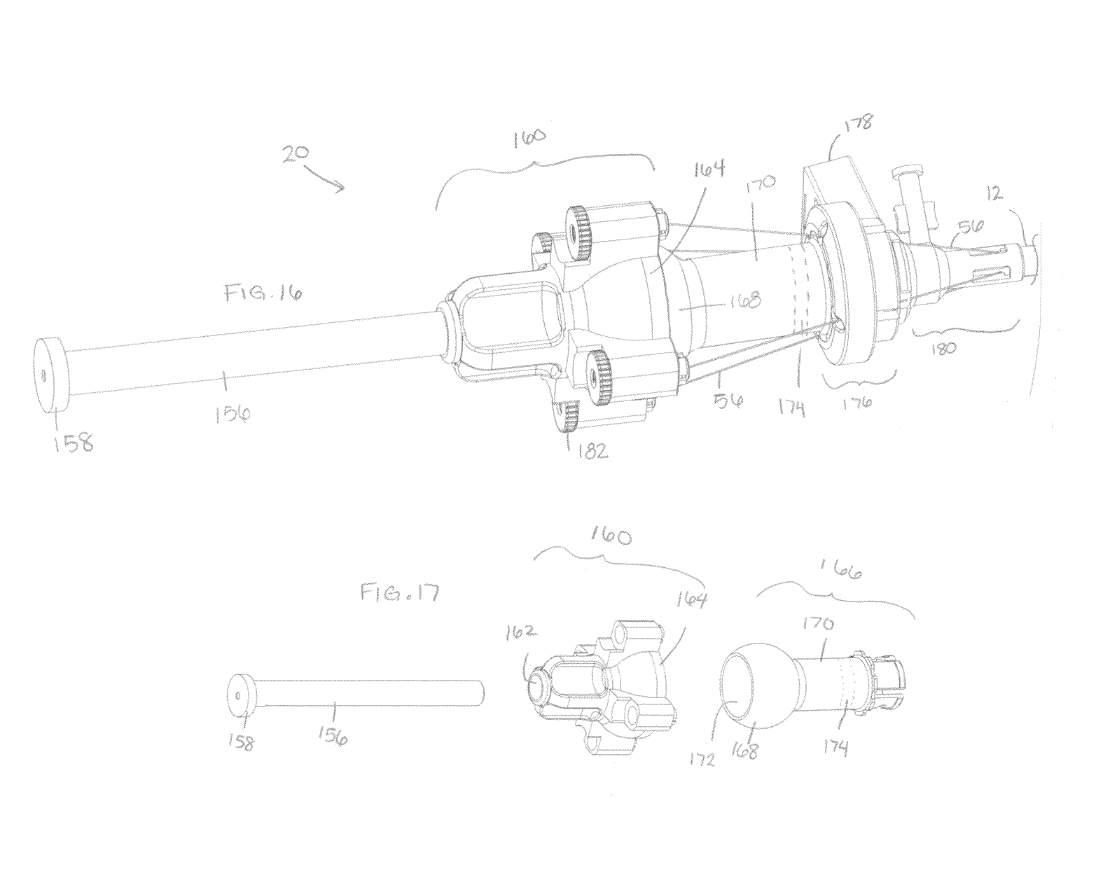

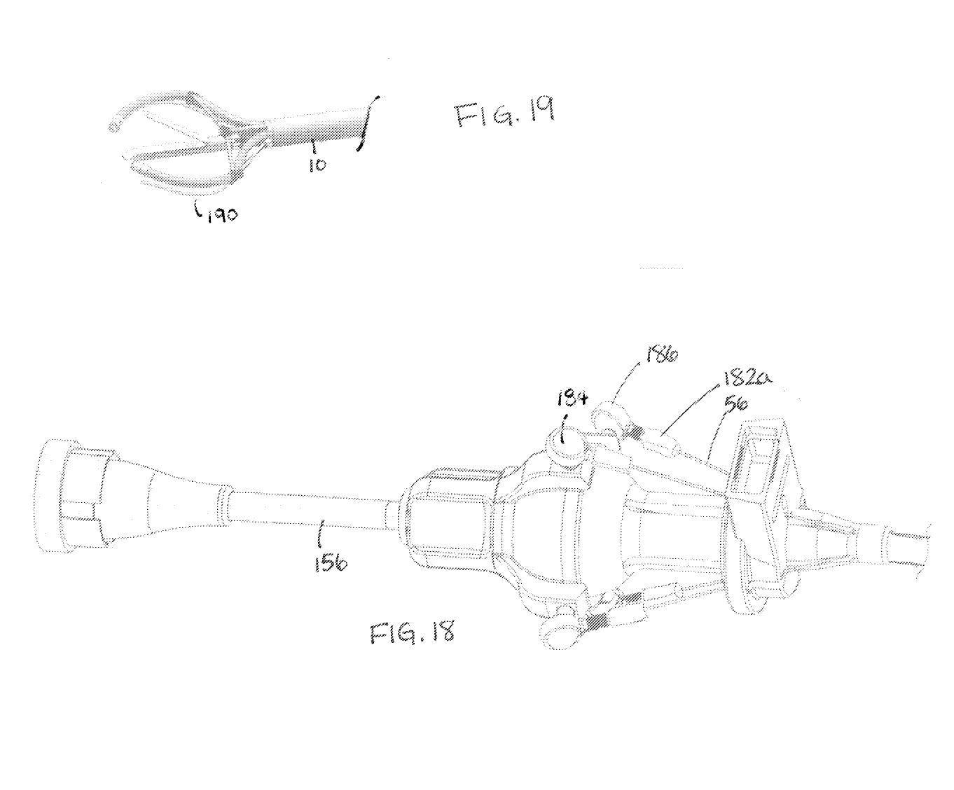

Details of the actuators will now be described with respect to FIG. 16. Each actuator 20 includes a control tube 156 having proximal entry port 158 for receiving a medical instrument. The control tube 156 preferably has a lubricious lining formed of PTFE or other suitable material so as to allow instruments inserted through the actuator to slide with ease. A proximal gimbal portion 160 is positioned distally of the control tube 156 and includes a proximal opening 162 which receives the distal end of the tube 156. The proximal gimbal portion 160 also includes a distally facing socket 164. A distal gimbal portion 166 includes a proximally facing ball 168 disposed within the socket 164 and a tubular housing 170 extending distally from the ball 168. The ball 168 has a proximally-facing opening 172 as shown in FIG. 17. A valve 174, which may be a cross-slit duck bill valve, is disposed within the tubular housing 170. The valve 174 functions to seal the actuator against loss of inflation pressure when no instruments are positioned through it.

The tubular housing 170 extends into a coupler 176 having a mount 178 used to attach the actuator 20 to the frame 22 (see FIG. 1). An end fitting 180 extends between the coupler 176 and the instrument delivery tube 12.

The pullwires 56 exiting the proximal end of each instrument delivery tube 12 pass through the sidewalls of the corresponding end fitting 180, through openings in the coupling 186, and into engagement with nuts 182 mounted to the proximal gimbal section 160. Nuts 182 may be adjustable to increase or decrease the amount of tension on the pullwires 56. In a slight modification to the FIG. 16 embodiment, nuts 182 are replaced by ball pivot mounts 184 to create a universal joint for each pullwire. Each pullwire 56 is attached by a tensioning nut housing 182a to a ring 186 that encircles the corresponding ball pivot mount 184 and that has full freedom to move in any direction over the surface of the ball pivot.

During use of the actuation system, the shaft of an instrument I extends through the control tube 156, proximal gimbal portion 182, distal gimbal portion etc. and through the instrument delivery tube 12 such that its operative end is disposed within the body cavity as shown in FIG. 1B. A suitable instrument will have a rigid proximal section that will be disposed within or otherwise in contact with the control tube 156, and a flexible distal section. When it becomes necessary for the surgeon to articulate the distal end of the instrument I, s/he intuitively moves the handle of that instrument, causing the control tube 156 to move with it. The socket of proximal gimbal portion 160 will move over the ball surface of the distal gimbal portion 166, thus tensioning the pullwires in accordance with the angle of the proximal gimbal portion relative to the distal gimbal portion. The distal portion of the instrument will deflect accordingly as a result of the action of the gimbal on the pullwires of the instrument delivery tube. Thus if it is desired to raise the distal end of the instrument, the user will lower the handle, moving the gimbal socket 164 downwardly over the gimbal ball 168. This will thus apply tension to the upper pullwire 56, causing upward deflection of the instrument delivery tube as well as the distal end of the instrument. Lateral movement of the instrument shaft to the right will tension the corresponding side pullwire to cause the distal portion of the instrument delivery tube to bend to the left. The actuator system allows combinations of vertical and lateral deflection, giving 360° deflection to the instrument delivery tube. The user may additionally advance/retract the tool longitudinally within the instrument delivery tube, and/or axially rotate the instrument within the instrument delivery tube when required.

Additional embodiments of actuators are shown and described in co-pending application Ser. No. 12/511,043 (Attorney Docket: TRX-2220), entitled MULTI-INSTRUMENT ACCESS DEVICES AND SYSTEMS, filed Jul. 28, 2009, which is incorporated herein by reference for all purposes.

Insufflation/Smoke Evacuation

In addition to the luer valve 48 described in connection with FIG. 3A, the system may include additional luer valves also suitable for coupling to a source of insufflation gas for insufflating the abdominal cavity through the insertion tube 10 or to a vacuum source for use in evacuating smoke from the abdominal cavity during electrosurgical or other thermal procedures. As shown in FIG. 16, such additional luer valves 48a may be positioned in one or both of the actuators 20, such as in the distal fittings 180 as shown in FIG. 16, allowing insufflation or smoke evacuation to be achieved through the instrument delivery tubes 12. The valves may also be opened to passively evacuate smoke from the abdominal cavity.

In an alternative embodiment shown in FIG. 19, auxiliary insufflation/smoke evacuation tubes 190 may extend from the insertion tube 10. In this embodiment, luer valves (not shown) at the proximal end of the system are fluidly coupled to the auxiliary tubes 190.

System Mounting

Referring to FIG. 20A, an operating room fixture such as a table-mounted arm 200 (see also FIG. 1B) is provided for supporting the system 100 in proximity to the patient, without requiring the system 100 to be held in place by operating room personnel. The operating room fixture includes a clamp 192 which is designed to be coupled to the system's spherical mount 26, allowing the system 100 to be oriented in an unlimited number of positions. In the illustrated embodiment, the clamp 192 comprises a collar having semi-annular segments 194. Each segment 194 includes a first end 197 coupled to the other one of the segments, and a second end 198 hinged to a latch 196. The collar has an unlatched position shown in FIG. 20A in which the latch 196 is pivoted outwardly to separate the ends 198 of the semi-annular segments. The latch is inwardly pivotable to place the collar in a latched position, in which the ends 198 are drawn closer together and retained in the closed position by the latch 196.

To mount the system to the fixture 200, the collar is placed in the unlatched position and disposed around the spherical mount 46. The user places the system 100 in the desired three-dimensional orientation and then closes the latch 196 to place the collar 192 in the latched position, thereby capturing the spherical mount 46 between them. If at any time during the procedure the user wishes to adjust the orientation of the system, s/he may unlatch the collar 192 to do so. Given the universal nature of the coupling between the collar 192 and the spherical mount 46, the user may chose to alter the pitch, roll and/or yaw relative to the longitudinal axis of the system and remain able to couple the repositioned system to the collar 192.

System Use

In an exemplary procedure using the disclosed system, the system is introduced through an opening (e.g. an incision or puncture) formed in a body wall, namely through the skin and underlying tissue, to give access to a body cavity such as the peritoneal cavity. In some procedures, the opening may be formed through the umbilicus for purposes of cosmesis. Given the small diameter of the main tube 12 (which may be less than 30 mm, and which in preferred embodiments is approximately 25 mm or less), only a single small incision is needed.

Initially, the deployment system is placed in the collapsed position shown in FIG. 7A, with the link arms 68 generally parallel as shown, to position the instrument delivery tubes in a streamlined orientation for passage through the opening in the body wall. In this position, the struts 78 are also parallel to one another and to the link arms 68.

A protective sheath may be positioned covering the distal portion of the device (e.g. the instrument delivery tubes and link arms and the main tube) to allow atraumatic passage through the incision and into the underlying cavity, and then removed from the system for deployment of the instrument delivery tubes. The body cavity is inflated via the insertion tube or access tubes using a source of insufflation gas coupled to the system via one or more of the luer valves on the system.

The rigid tube 14 is advanced distally using the ring 84 or other actuation device to expand the link arms 64 (or pivot the spring members of FIG. 9A) to move the instrument delivery tubes to the expanded position shown in FIG. 7B. The ratchet system associated with the ring 84 locks the longitudinal position of the rigid tube 14, thus maintaining the lateral separation of the instrument delivery tubes until repositioning is desired. If a change in the lateral spacing is needed during the procedure, the user disengages the ratchet system by depressing the trigger 86, allowing the separation distance between the instrument delivery tubes to be increased or decreased.

Flexible instruments are advanced into the body cavity via the actuation system and instrument delivery tubes 12, such that the handles of the instruments are disposed proximally of the control tubes 156. The instruments are used to perform the procedure (e.g. cutting, manipulation, cauterization of tissue) within the body cavity. Instruments suitable for use with the system include those described in co-pending U.S. application Ser. No. 12/511,053, filed Jul. 28, 2009, (Attorney Docket No. TRX-2110), entitled Flexible Dissecting Forceps, and U.S. application Ser. No. 12/511,050, filed Jul. 28, 2009, (Attorney Docket No. TRX-2400), entitled Flexible Medical Instruments, each of which is incorporated herein by reference. The actuators 20 are used to steer the instruments within the body cavity as described above.

Where electrosurgical procedures are performed, smoke evacuation may be carried out through the system as described above.

Rigid or flexible instruments may be introduced into the rigid tubes 14, 16, and will have their proximal ends or handles disposed proximally of the frame 22 for access by the user. In some procedures, a rigid endoscope may be positioned in upper rigid tube 14 or lower rigid tube 16 and used to observe the procedure being carried out using the body cavity. Clamp assembly 118 is used as described above to fix the longitudinal position of a rigid instrument in the upper rigid tube 14.

While certain embodiments have been described above, it should be understood that these embodiments are presented by way of example, and not limitation. It will be apparent to persons skilled in the relevant art that various changes in form and detail may be made therein without departing from the spirit and scope of the invention. This is especially true in light of technology and terms within the relevant art(s) that may be later developed.

Any and all patents, patent applications and printed publications referred to above are incorporated herein by reference.

We claim:

1. An access system for surgical procedures, the access system comprising:
an insertion tube;
a pair of flexible instrument delivery tubes extending through the insertion tube;
a rigid tube extending through the insertion tube;
a pair of link arms, each having a distal end pivotally coupled to an instrument delivery tube and a proximal end pivotally coupled to the rigid tube, wherein the rigid tube is longitudinally slidable relative to the insertion tube between a proximal position and a distal position, wherein movement of the rigid tube from the proximal position to the distal position causes the distal ends of the link arms to pivot laterally outwardly relative to the rigid tube.

2. The access system according to claim 1, further including a lock having a first member coupled to the rigid tube and a second member coupled to the insertion tube, wherein the lock has a locked position in which the first member is engaged with the second member to prevent longitudinal movement of the rigid tube relative to the insertion tube, and an unlocked position allowing longitudinal movement of the rigid tube relative to the insertion tube.

3. The access system according to claim 2, wherein the first member includes a catch and the second member includes a ratchet plate having a plurality of teeth, wherein in the locked position the catch is engaged with at least one of the teeth.

4. The access system according to claim 2, wherein the rigid tube includes a lumen, and wherein the access system includes a clamp moveable into contact with the shaft of an instrument disposed within the lumen to clamp the shaft against longitudinal movement of the instrument within the lumen.

5. The access system according to claim 4, wherein the lock and the clamp are arranged to permit longitudinal movement of an instrument in the rigid tube when the lock is in the locked position and the clamp is positioned out of contact with the shaft of the instrument.

6. The access system according to claim 4, wherein the lock and the clamp are arranged to permit longitudinal movement of the rigid tube relative to the insertion tube when the lock is in the unlocked position and the clamp is positioned to clamp the shaft against longitudinal movement of the instrument within the lumen.

7. The access system according to claim 1, wherein movement of the rigid tube from the proximal position to the distal position causes the distal ends of the link arms to change a lateral separation distance between instrument delivery tubes, and wherein the rigid tube is longitudinally moveable to selectively position the instrument delivery tubes at a desired lateral separation distance.

8. The access system according to claim 1, wherein the distal ends of the link arms remain distal to the proximal ends of the link arms during movement of the rigid tube from the proximal position to the distal position.

9. The access system according to claim 1, wherein the distal end of the rigid tube remains proximal to the distal ends of the instrument delivery tubes during movement of the rigid tube from the proximal position to the distal position.

10. The access system according to claim 1, wherein the instrument delivery tubes are positioned parallel to one another when the rigid tube is in the proximal position.

11. The access system according to claim 10, wherein the distal ends of the instrument delivery tubes are in longitudinal contact with one another when the rigid tube is in the proximal position.

12. The access system according to claim 1, further including a second rigid tube within the insertion tube.

13. The access system according to claim 1, wherein the rigid tube includes a lumen, and wherein the access system includes a clamp moveable into contact with the shaft of an instrument disposed within the lumen to clamp the shaft against longitudinal movement of the instrument within the lumen.

14. The access system according to claim 1, further including a pair of support members, each support member having a first end pivotally coupled to the insertion tube and a second end pivotally coupled to a corresponding instrument delivery tube.

15. The access system according to claim 14, wherein the distal ends of the link arms remain distal to the proximal ends of the link arms during movement of the rigid tube from the proximal position to the distal position.

16. The access system according to claim 14, wherein the distal end of a first one of the links and the distal end of a first one of the support members are coupled to the rigid tube for pivotable movement relative to a first common pivot axis, and wherein the distal end of a second one of the links and the distal end of a second one of the support members are coupled to the rigid tube for pivotable movement relative to a second common pivot axis.

17. The access system according to claim 16, further including a collar on each instrument delivery tube, wherein the corresponding link arm and support member are pivotally attached to the collar.

18. The access system of claim 1, further including a luer port in fluid communication with the insertion tube.

19. The access system of claim 1, further including a luer port in fluid communication with an instrument delivery tube

20. The access system of claim 1;
wherein the system further includes a base having a distal portion and a proximal portion;
wherein the insertion tube is mounted to the distal portion of the frame;
wherein each instrument delivery tube has a distal portion positioned distally of the insertion tube, and a proximal portion positioning proximally of the insertion tube;
and wherein the system further includes a pair of actuators, each positioned on the base and coupled to the proximal portion of a corresponding instrument delivery tube, each actuator moveable to deflect the distal portion of the corresponding instrument delivery tube.

21. The instrument access system of claim 20, wherein the base includes a first opening, the first opening at least partially aligned with the insertion tube to permit an instrument to be passed through the first opening and into the insertion tube; and
a clamp moveable into contact with the shaft of an instrument extending through the first opening to restrict longitudinal movement of the instrument relative to the insertion tube.

22. The instrument access system of claim 21, wherein the system further includes:
a rigid tube extending through the insertion tube, the rigid tube positioned in alignment with the opening in the base such that an instrument may be inserted through the opening and into the rigid tube.

23. The instrument access system of claim 21, wherein the base includes a second opening beneath the first opening, the second opening positioned for passage of a second instrument therethrough and into the insertion tube.

24. The instrument access system of claim 23, further including a second rigid tube within the insertion tube, the second rigid tube positioned to receive an instrument passed through the second opening.

25. The instrument access system of claim 20, wherein:
each actuator includes
a distal portion coupled to the corresponding wing section, the distal portion including a ball member;
a proximal portion including a socket disposed on the ball member; and
each instrument delivery tube includes a plurality of actuation cables extending therethrough, the actuation cables coupled to a corresponding one of the actuators at the proximal portion; and
each actuator includes an opening for passage of an instrument shaft through the proximal portion and through the distal portion and into the corresponding instrument delivery tube, such that movement of a proximal end of the instrument shaft causes corresponding movement of the socket on the ball member and corresponding activation of the actuation cables.

26. An instrument access system, comprising:
a base having a distal portion and a proximal portion;
an insertion tube mounted to the distal portion of the base;
a pair of flexible instrument delivery tubes extending through the insertion tube, each insertion tube having a distal portion positioned distally of the insertion tube, and a proximal portion positioning proximally of the insertion tube;
a pair of actuators in spaced apart positions on the base, each actuator coupled to the proximal portion of a corresponding instrument delivery tube, each actuator moveable to deflect the distal portion of the corresponding instrument delivery tube;
a support attachable to a patient support table; and
a mount including a spherical member coupled to the base, and a split ring coupled to the support, the split ring having an opening and being moveable between an open position in which the opening is proportioned to receive the spherical member, and a closed position in which, when the spherical member is disposed in the opening, the split ring engages the spherical member within the opening.

27. An access system for surgical procedures, the access system comprising:
an insertion tube having a distal end and a lumen;
a pair of instrument delivery tubes extending through the lumen of the insertion tube, each instrument delivery tube having a fixed longitudinal position relative to the insertion tube, each instrument delivery tube including a steerable distal portion positioned distal to the distal end of the insertion tube and a proximal actuator moveable to steer the distal portion;
a first rigid tube mounted within the lumen and having a distal end disposed within the lumen;
a second rigid tube having a fixed shape, the second rigid tube having a distal end positioned distal to the distal end of the insertion tube.

28. The system of claim 27, wherein the longitudinal axes of the instrument delivery tubes lie in a first plane, and wherein the longitudinal axes of the first and second rigid tubes lie in a second plane transverse to the first plane.

29. The system of claim 28 wherein the first and second planes are generally perpendicular to one another.

30. The system of claim 27, further including a pair of link arms, each having a distal end pivotally coupled to an instrument delivery tube and a proximal end pivotally coupled to the second rigid tube, wherein the second rigid tube is longitudinally slidable relative to the insertion tube between a proximal position and a distal position, wherein movement of the rigid tube from the proximal position to the distal position causes the distal ends of the link arms to pivot laterally outwardly relative to the rigid tube.

31. The system of claim 1, wherein when the link arms and the support members are parallel to one another when the rigid tube is in the proximal position.

32. The system of claim 27, wherein the instrument delivery tubes are of unequal diameter.

33. The system of claim 27 wherein the first and second rigid tubes are of unequal diameter.
